(19) United States
(12) Patent Application Publication
(10) Pub. No.: US 2006/0035356 A1
(43) Pub. Date: Feb. 16, 2006

(54) Introduction of microorganisms in bio-assisted heap leaching operations

(76) Inventors: Frank K. Crundwell, Parkwood (ZA); Alan E. Norton, Brackendowns (ZA)

Correspondence Address: DARBY & DARBY P.C., P. O. BOX 5257, NEW YORK, NY, 10150-5257, US

(21) Appl. No.: 10/528,381

(22) PCT Filed: Sep. 15, 2003
(86) PCT No.: PCT/IB03/04186

§ 371 Date: Jun. 1, 2005

(30) Foreign Application Priority Data
Sep. 17, 2002 (ZA) 2002/7439

(51) Int. Cl. C12P 3/00

(52) U.S. Cl. 435/168

(57) ABSTRACT

The invention discloses a method of introducing microorganisms into a heap of material for bio-assisted heap leaching by prepraring microorganisms substantially without exopolymers on their external cell walls; adding such microorganisms to the heap; and re-activating the production of exopolymers on the external cells walls of the microorganisms in the heap. The invention also extends to a method of enriching the environment of microorganisms' embedded in a heap for bio-assisted heap leaching.

### FIELD OF THE INVENTION

This invention relates to bio-assisted heap oxidation and leaching for the recovery of metals from ore.

### BACKGROUND TO THE INVENTION

Bio-assisted heap and dump leaching occupies an increasingly important position in the recovery of metals from ores. The recovery of metals is carried out commercially on copper, nickel, and uranium ores and as a pre-treatment process for the recovery of gold.

It is well known that the presence of bacteria or achae in the heaps is essential for the effective operation of these heaps. Microorganisms act as catalysts in the oxidation reactions, thus accelerating the dissolution of the minerals. The establishment of a viable population of microorganisms within the heap is essential for the effective operation of these heaps. In practice, bacteria are not always specifically introduced into the heap at the start of the operation. This results in poor performance of the heap because of the lack of the essential catalyst.

Microorganisms can be introduced into the heap during the formation of the heap. The introduction of microorganisms during the formation of the heap of crushed material is not effective because of the common practice of mixing acid with the crushed ore. This acid destroys the microorganisms, rendering their introduction ineffective.

The introduction of microorganisms into heaps after they have been formed is not effective. It is well known that bacteria have adhesive material on their surfaces that cause them to adhere to surfaces. Porous material such as that presented in an ore heap or dump has a high surface area per unit volume. Microorganisms introduced through the irrigation of the heap will rapidly attach to the ore surfaces at the top of the heap and will fail to penetrate into the bulk of the heap. The adhesion of microorganisms has generally been found to be irreversible, with the result that even with time the microorganisms are unlikely to penetrate in sufficiently large numbers into the bulk of the heap. In confirmation of this prior knowledge, MacLeod et al. (In “Plugging of a model rock system by using starved bacteria”, Applied and Environmental Microbiology, 1988, volume 54, pp 1365-1372) found that the penetration of microorganisms was significantly diminished within a few centimeters into the porous media. In addition, U.S. Pat. No. 6,383,458 states that the microorganisms are concentrated only in the top one or two feet of a heap when the method of introduction is by a solution containing microorganisms that sprinkled or dripped on to the heap.

Therefore the introduction of microorganisms to the heap once the heap has been constructed results in a ‘skin’ of microorganisms at the top of the heap or dump without the effective penetration of the bacteria throughout the heap.

Since heaps are in most cases more than 6 meters deep, the efficiency of bacterial penetration is extremely low. The prior art methods of introduction of microorganisms into the heap results in poor dispersal and distribution, with the result that dead regions may arise in the heap, resulting in poor extractions of metals from the ore.

Further practical difficulties arise when the heap or dump is stacked using trucks, such as the stacking of uncrushed ore on a residue dump. The effective introduction of microorganisms in this case can only performed by the Irrigation of the ore material, which gives rise to the problem of the formation of ‘skin’ of microorganisms rather than their penetration through the depth of the heap or dump.

Further difficulties arise if the temperature in the heap rises. This is a desirable state, since the rate of the leaching reactions is dependent on temperature. The higher the temperature, the greater is, the rate of leaching. However, the microorganisms have specific ranges of temperature in which they are effective. Indeed, temperatures above a critical temperature will result in the death of the microorganisms. As the temperature rises in the heap, it will be necessary to introduce microorganisms that are best suited to catalyzing the oxidation processes at those temperatures. However, because of the problem of the formation of ‘skin’ layer mentioned above, the introduction of these microorganisms will not be effective. Since a temperature rise may cause the death of the microorganisms and the subsequent introduction of microorganisms is inefficient, there is the possibility of the failure of the operation of the heap.

### OBJECT OF THE INVENTION

It is an object of this invention to provide a process that at least partly alleviates some of the abovementioned problems.

### SUMMARY OF THE INVENTION

In accordance with this invention there is provided a method of introducing microorganisms into a heap of material for bio-assisted heap leaching comprising:
a) preparing microorganisms without exopolymers on their external cell walls;
b) adding microorganisms prepared according to step a) to the heap;
c) assisted or un-assisted re-activation of the production of exopolymers on the external cells walls of the microorganisms in the heap.

There is further provided for step a) to include exposing the microorganisms to a low nutrient environment or starving the microorganisms.

There is still further provided for the microorganisms to be starved by limiting the amount of carbon available for the microorganisms.

There is also provided for step b) to include one or more of adding microorganisms to the heap during formation thereof, drip irrigation of the heap, sprinkling of the heap, and pressurized irrigation of the heap.

There is further provided for the assisted re-activation to comprise exposing the microorganisms to a nutrient rich environment, including:

a) embedding solid nutrients in the heap, and preferably for the solid nutrients to be slow release nutrients;

b) irrigating the heap with a nutrient rich solution;

c) aerating the heap with nutrient rich gas, preferably one or more of a nutrient aerosol and ammonia; and

d) aerating the heap with a gas enriched In carbon dioxide.

There is also provided for un-assisted re-activation to include re-activation due to one or more of prevalent conditions in the heap, and natural gas flow through the heap including flow of carbon dioxide through the heap.

### DETAILED DESCRIPTION OF THE INVENTION

The invention is described below by way of example only and with reference to Example 1, which is a preferred embodiment of the invention.

The present invention provides a method of preparation of microorganisms in such a manner that they are able to penetrate the depth of the heap or dump when introduced to the heap by irrigation of a solution containing the prepared microorganisms at the top the heap.

This is achieved by preparing the microorganisms in a state in which the microorganisms do not produce exopolymers on their external cell walls, which generally requires their preparation in a low nutrient environment.

In the present application, the microorganisms are starved to the point where the microorganisms decrease production of exopolymers on their external cell walls by lowering nutrients In a growth medium of the microorganism. This renders the cells non-adhesive, and suitable for the introduction into the heap or dump.

The microorganisms commonly found in bio-assisted leaching operations are autotrophic. The creation of a carbon-free growth medium requires the limitation of carbon dioxide dissolved in the growth medium.

The non-adherent microorganisms are introduced onto the heap by irrigation of the heap with a solution rich in the prepared microorganisms and allowed to penetrate the depth of the heap or dump. Once in the heap, the microorganism are activated or rendered adhesive either naturally through the change In environment, for instance due to the presence of carbon dioxide in the atmosphere of the heap, or by the Irrigation of the heap with a nutrient rich solution causing the prepared microorganisms to develop exopolymers and to adhere to the external surfaces of ore particles in the heap.

Subsequent to the establishment of a population of microorganisms in the heap or dump, nutrients are supplied to the microorganisms introduced into the heap by means of the irrigation solution of the heap, or adding slow release nutrient solids to the ore or alternatively by means of aerating the heap with a nutrient aerosol and/or ammonia gas, as well as adding a carbon source via carbonate mixed with the ore or carbon dioxide added to the aeration supply.

The rate of dissolution of minerals is dependent on the catalytic action of microorganisms in the heap. In both the start-up phase and the operational phase of the heap operation, these microorganisms play a critical role.

The microorganisms will not penetrate the depth of the heap simply by irrigating the heaps with a solution enriched with these microorganisms. Rather, they will adhere to the rocks and minerals at the point of irrigation or injection, thus forming a “skin” of microorganisms at the surface of the heap. This is because the external cell walls of the microorganisms are coated with exopolymers that are adhesive. In fact, this property of the adhesive nature of microorganisms is the basis for the effectiveness of the removal of bacteria by sand filters for the purification of water.

Thus it is very difficult to disperse microorganisms that are in their normal vegetative state throughout the heap simply by irrigating the heap with inoculum; at finer particle sizes it will become virtually impossible.

However, microorganisms that have been specially treated to reduce the production of polymeric material on the external surface of their cell walls will penetrate the heap and will not adhere to the mineral and rock surfaces of the heap. Such a preparation of microorganisms will enable the uniform dispersal and distribution of microorganisms within the heap. Once the solution enriched in microorganisms that lack adhesive coatings has fully penetrated the heap body of the heap, the adhesive properties of the microorganisms can be restored.

Microorganisms that lack the adhesive coatings can be prepared by limiting the supply of nutrients to the microorganisms.

The limitation of the nutrient supply to the microorganisms is referred to as ‘starvation’ of the cells. Starvation will result in their lowering of the production of adhesive polymer coatings (exopolymers) on the cell walls of the microorganisms. Other preparations may be by the formation of spores or by the formation of ultramicrobacteria (UMB). In these states, it is known that the microorganisms do not produce polymers on their external cell walls. The important property of this starvation treatment for this invention is not the production of ultramicrobacteria or spores, but that the preparation of microorganisms that do not adhere to porous media, so that they can be effectively introduced to and dispersed within a heap.

The production of microorganisms with reduced exopolymers by starvation is achieved most often by reducing the carbon source. In the case of microorganisms suitable for heap leaching, the carbon source is often carbon dioxide dissolved in solution. Preparation of the non-adhesive cells can be achieved by removing carbon dioxide from the solution or limiting the concentration of dissolved carbon dioxide, such as by removing carbon dioxide from the air source required for the growth of the microorganisms, or by using pure oxygen and nitrogen in the gas supply to the growth culture. The reduction of the exopolymers may also be achieved by limiting a nutrient other than the source of carbon. The non-adhesive cells may also be prepared by transferring them to a low nutrient environment.

The resuscitation of the adhesive properties of the microorganisms is achieved either by providing the microorganisms with nutrients, or by allowing the microorganisms to restore this property due to the conditions present in the heap.

Therefore this invention concerns the method of preparing microorganisms in reactor by a suitable starvation method, injecting them into the heap or dump, and then resuscitating them, either by injecting a nutrient rich solution into the heap or dump, or by allowing the microorganisms to naturally revert back to their adhesive state. Using this aspect of the invention it will be possible to re-inoculate a heap or dump during it's operating life. For example, failure of appropriate controls may result in the introduction of toxic substances or elevated temperatures that poison or kill the microorganisms; this invention could be used to re-inoculate the heap after such an event and resume leaching thereafter. Additionally this aspect of the Invention could be used to re-inoculate old heaps or dumps, leach the heap or dump and extract further values from them.

In accordance with this invention, the microorganisms that have been found to be important in bio-assisted heap leaching are autotrophic bacteria and archae belonging, but not limited to the genera Thiobacillus, Acidothiobacillus, Leptospirillum, Sulfolobus, Acidianus, Metallosphaera. Both the processes for the growth and the starvation of the microorganisms can be performed continuous, semi-continuous, fed-batch or batch reactors.

Once the population has been established in the heap by this invention, the microorganisms must have an adequate supply of nutrients to maintain a healthy microbial environment. In tank systems processing concentrates the nutrients are added continuously with the concentrate. In the case of heap leaching though, nutrients in solid form can only be added once, when the ore is stacked. Such nutrients should be specifically designed to release slowly into solution, for the entire duration of the leach cycle. Alternatively the nutrients can be added with the irrigation solution, although in high heaps in particular, chemistry considerations may make it difficult for nutrients to reach the lower part of the heap. It is also foreseen that nutrients can be added via air addition as an aerosol and/or ammonia gas. Additionally the microorganisms require a source of carbon for cell growth. Carbon can be conveniently supplied by carbonates in the ore or by adding carbonates mixed in with the ore heap or by adding carbon dioxide to the aeration supply. The amount of carbon and other nutrients added is chosen to maintain high rates of microorganism growth and sulphide oxidation. In particular, carbon supply must be adequate when the microbial populations are under establishment at the beginning of the cycle and when temperature shifts into the regions where moderate thermophile microorganisms and thermophile microorganisms become active. Bouffard and Dixon (In S. C. Bouffard and D. G. Dixon, On the rate-limiting steps of pyritic refractory gold ore heap leaching: Results from small and large column tests, Minerals Engineering, Vol. 15, no. 11, 2002) indicate a carbon requirement of about 0.2 g per kg of ore in the bacterial growth phase. Supplementing the air with addition of carbon dioxide gas amounting to between 0 and 5% of the volumetric gas flow, at the appropriate time in the leach cycle, or adding sufficient carbonate to the ore will likely be the best means of meeting this requirement.

The maintenance of the microbial population in the heap or dump may require the removal of residual solvent extraction organic, iron, as well as toxic elements and organics either substantially to promote high microbial activity, with high ferrous-to-ferric conversion; or in part to reduce ferrous-to-ferric conversion to achieve a lower redox potential within the heap.

### EXAMPLE 1

The required microbial population is selected on the basis of the conditions expected in the heap. For example, at the start up phase of the heap leaching cycle, the temperatures in the heap are expected to be below 45° C. Moderate thermophiles, thermophiles or extreme thermophiles may be selected for operation at higher temperatures. It is preferable to select at least two species, one that oxidizes ferrous sulphate to ferric sulphate, and another that oxidizes reduced sulphur species to sulphate, unless the microorganism selected is capable of oxidizing both ferrous sulphate and reduced sulphur. The selected microorganisms are grown either together in a single reactor or separately in different reactors. The concentration of nutrients in the growth medium in these reactors must be controlled such that concentration of the final or exit solution is at a minimum.

The solution enriched in these microorganisms is processed either directly with the supernatant from the growth reactor, or by removing the supernatant from the growth reactor by an operation such as centrifuging. The microorganisms, either with the supernatant or without it, are added to the starvation reactor. The starvation reactor

1. A method of introducing microorganisms into a heap of material for bio-assisted heap leaching comprising:
a) preparing microorganisms substantially without exopolymers on their external cell walls;
b) adding microorganisms prepared according to step a) to the heap;
c) at least one of assisted or un-assisted re-activation of the production of exopolymers on the external cells walls of the microorganisms.

2. A method as claimed in claim 1 in which step a) includes exposing the microorganisms to a low nutrient environment or starving the microorganisms.

3. A method as claimed in claim 2 in which the microorganisms are starved by limiting the amount of carbon available to the microorganisms.

4. A method as claimed in claim 1 in which step b) includes one or more of adding microorganisms to the heap during formation thereof, drip irrigation of the heap, sprinkling of the heap, and pressurized irrigation of the heap.

5. A method as claimed in claim 4 in which the assisted re-activation comprises exposing the microorganisms to a nutrient rich environment.

6. A method as claimed in claim 5 in which the step of exposing the microorganisms to a nutrient rich environment includes one or more of:
a) embedding solid nutrients in the heap;
b) irrigating the heap with a nutrient rich solution;
c) aerating the heap with nutrient rich gas; and
d) aerating the heap with a gas enriched in carbon dioxide.

7. A method as claimed in claim 6 in which includes the step of embedding a carbon source in the heap.

8. A method as claimed in claim 7 in which the carbon source comprises carbonate.

9. A method as claimed in claim 6 in which the solid nutrients of step a) comprises slow release nutrients.

10. A method as claimed in claim 6 in which the gas of the step c) is enriched with one or more of a nutrient aerosol or ammonia.

11. A method as claimed in claim 1 in which the assisted re-activation comprises exposing the microorganisms to a nutrient rich environment.

12. A method as claimed in claim 11 in which the step of exposing the microorganisms to a nutrient rich environment includes one or more of:
a) embedding solid nutrients in the heap;,
b) irrigating the heap with a nutrient rich solution;
c) aerating the heap with nutrient rich gas; and
d) aerating the heap with a gas enriched in carbon dioxide.

13. A method as claimed in claim 12 which includes the step of embedding a carbon source in the heap.

14. A method as claimed in claim 13 in which the carbon source comprises carbonate.

15. A method as claimed in claim 12 in which the solid nutrients of step a) comprises slow release nutrients.

16. A method as claimed in claim 12 in which the gas of the step c) is enriched with or more of a nutrient aerosol or ammonia.

17. A method as claimed in claim 1 in which the un-assisted re-activation includes re-activation due to one or more of prevalent conditions in the heap and natural gas flow through the heap.

18. A method as claimed in claim 17 in which the natural gas includes carbon dioxide.
